(19) United States
(12) Patent Application Publication
(10) Pub. No.: US 2023/0293151 A1
(43) Pub. Date: Sep. 21, 2023

(54) ULTRASONIC DIAGNOSIS APPARATUS AND ELECTROCARDIAC WAVEFORM PROCESSING METHOD

(71) Applicant: FUJIFILM Healthcare Corporation, Kashiwa-shi (JP)

(72) Inventor: Satoshi Kibe, Chiba (JP)

(21) Appl. No.: 18/117,324

(22) Filed: Mar. 3, 2023

(30) Foreign Application Priority Data
Mar. 16, 2022 (JP) 2022-040877

(51) Int. Cl. A61B 8/00; A61B 8/06; A61B 5/339; A61B 5/347

(52) U.S. Cl. CPC A61B 8/463; A61B 5/339; A61B 5/347; A61B 8/06

(57) ABSTRACT

A definer defines heartbeat segments in an electrocardiac waveform. A primary determiner determines, from among the heartbeat segments, segments that satisfy a primary determination criterion as provisional stable segments. A secondary determiner determines a provisional stable segment as a stable segment when the provisional stable segment satisfies a secondary determination criterion. An end-diastole detector and an end-systole detector detect an end-diastole and an end-systole in a segment of interest. At the time of changing the segment of interest, a selection control unit controls selection of segment of interest such that the segment of interest after change corresponds to a stable segment.

### CROSS REFERENCE TO RELATED APPLICATION

This application claims priority to Japanese Patent Application No. 2022-040877 filed on Mar. 16, 2022, which is incorporated herein by reference in its entirety including the specification, claims, drawings, and abstract.

### TECHNICAL FIELD

The present disclosure relates to an ultrasonic diagnosis apparatus and an electrocardiac waveform processing method, and more particularly to a technique of detecting end-diastole and end-systole.

### BACKGROUND

Ultrasonic diagnosis apparatuses used for ultrasonic examination of the heart generally have the function of obtaining an electrocardiac signal (i.e., an ECG signal). In such ultrasonic diagnosis apparatuses, a waveform representing the electrocardiac signal (i.e., an electrocardiac waveform) is displayed together with an ultrasound image. The electrocardiac waveform is composed of a plurality of heartbeat waveforms that are consecutive on a time axis. In a freeze state after a real-time operation, an ultrasound image corresponding to a time phase of the electrocardiac waveform designated or selected by the user (i.e., a time phase of interest) is displayed on a display.

Various measurements are carried out in order to evaluate the function of the heart. As a typical measurement value (i.e., a cardiac function evaluation value), the ejection fraction is known. The ejection fraction is generally calculated from the left ventricular volume at end-diastole and the left ventricular volume at end-systole. Each of these volumes is determined based on areas of the left ventricle in tomographic images. When the accuracy of identifying end-diastole and end-systole is low, the reliability of the calculated ejection fraction is decreased.

Document 1 (WO 2012/029616 A) and Document 2 (WO 2013/105568 A) disclose an ultrasonic diagnosis apparatus having a function of determining a stable heartbeat among a plurality of heartbeats as a stable segment. Document 3 (WO 2012/023399 A) discloses an ultrasonic diagnosis apparatus having a function of determining end-diastole and end-systole. Documents 1, 2, and 3 do not describe any technique of correlating the function of determining a stable segment and the function of determining end-diastole and end-systole.

The present disclosure is directed to providing an ultrasonic diagnosis apparatus and an electrocardiac waveform processing method that are capable of detecting end-diastole and end-systole in appropriate heartbeat segments.

### SUMMARY

An ultrasonic diagnosis apparatus according to an aspect of the present disclosure includes: an image forming unit configured to form an ultrasound image based on reception data obtained from an examinee; a defining unit configured to define, in units of heartbeat, a plurality of segments in an electrocardiac waveform representing an electrocardiac signal obtained from the examinee; a detection unit configured to detect, based on the electrocardiac waveform, an end-diastole and an end-systole in a segment of interest among the plurality of segments; and a display processing unit configured to display an end-diastole marker indicating the end-diastole in the segment of interest and an end-systole marker indicating the end-systole in the segment of interest in a case where the electrocardiac waveform is displayed together with the ultrasound image. The ultrasonic diagnosis apparatus further includes: a determining unit configured to determine, from among the plurality of segments, each segment that satisfies a stability criterion as a stable segment; and a selection control unit configured to, when a manipulation for changing the segment of interest is performed, control selection of a segment of interest after change such that the segment of interest after change is selected from among stable segments determined by the determining unit. When a manipulation for changing the segment of interest is performed, the detection unit detects an end-diastole and an end-systole in a specific stable segment, which is the segment of interest after change.

An electrocardiac waveform processing method according to an aspect of the present disclosure includes: a step of defining, in units of heartbeat, a plurality of segments in an electrocardiac waveform representing an electrocardiac signal obtained from an examinee; a step of detecting, based on the electrocardiac waveform, an end-diastole and an end-systole in a segment of interest among the plurality of segments; and a step of displaying an end-diastole marker indicating the end-diastole and an end-systole marker indicating the end-systole in a case where the electrocardiac waveform is displayed together with an ultrasound image. The method further includes: a step of determining, from among the plurality of segments, each segment that satisfies a stability criterion as a stable segment; and a step of, when a manipulation for changing the segment of interest is performed, controlling selection of a segment of interest after change such that the segment of interest after change is selected from among the determined stable segments. When a manipulation for changing the segment of interest is performed, an end-diastole and an end-systole are detected in a specific stable segment, which is the segment of interest after change.

### BRIEF DESCRIPTION OF DRAWINGS

Embodiment(s) of the present disclosure will be described based on the following figures, wherein:

FIG. 1 is a block diagram showing an example configuration of an ultrasonic diagnosis apparatus according to an embodiment;

FIG. 2 is a diagram showing an example configuration of a waveform analysis unit;

FIG. 3 is a diagram showing changes in a waveform image that are displayed at the time of a change of segment of interest;

FIG. 4 is a diagram showing a small number of provisional stable segment determination methods;

FIG. 5 is a diagram showing a first example of provisional stable segment determination;

FIG. 6 is a diagram showing a second example of provisional stable segment determination;

FIG. 7 is a diagram showing detection of an end-diastole and an end-systole in a stable segment;

FIG. 8 is a schematic diagram showing a stable segment determination method;

FIG. 9 is a diagram for explaining a plurality of options that can be selected;

FIG. 10 is a diagram showing an example operation according to an option 2 and an example operation according to an option 3;

FIG. 11 is a flowchart showing an example operation of the ultrasonic diagnosis apparatus; and

FIG. 12 is a diagram showing an example dual screen display.

### DESCRIPTION OF EMBODIMENTS

Embodiments will now be described by reference to the drawings.

### Overview of Embodiments

An ultrasonic diagnosis apparatus according to an embodiment includes an image forming unit, a defining unit, a detection unit, a display processing unit, a determining unit, and a selection control unit. The image forming unit forms an ultrasound image based on reception data obtained from an examinee. The defining unit defines, in units of heartbeat, a plurality of segments (i.e., heartbeat segments) in an electrocardiac waveform representing an electrocardiac signal obtained from the examinee. The detection unit detects, based on the electrocardiac waveform, an end-diastole and an end-systole in a segment of interest among the plurality of segments. The display processing unit displays an end-diastole marker indicating the end-diastole in the segment of interest and an end-systole marker indicating the end-systole in the segment of interest in a case where the electrocardiac waveform is displayed together with the ultrasound image. The determining unit determines, from among the plurality of segments, each segment that satisfies a stability criterion as a stable segment. When a manipulation for changing the segment of interest is performed, the selection control unit controls selection of a segment of interest after change such that the segment of interest after change is selected from among stable segments determined by the determining unit. When a manipulation for changing the segment of interest is performed, the detection unit detects an end-diastole and an end-systole in a specific stable segment, which is the segment of interest after change.

An ultrasonic diagnosis apparatus according to an embodiment comprises a processor. The processor functions as the above-noted image forming unit, defining unit, detection unit, display processing unit, determining unit, and selection control unit. The processor may be composed of one or a plurality of physical processors.

According to the above configuration, detection of an end-diastole and an end-systole in a stable segment is ensured after a change of segment of interest. Even when the segment of interest is repeatedly changed, detection of an end-diastole and an end-systole are to be always performed in a stable segment. Accordingly, the reliability of the detected end-diastole and end-systole can be enhanced.

When the time phase detection function is automatically activated at a point of transition to a freeze state, an end-diastole and an end-systole may be detected in the latest segment regardless of whether or not the latest segment serving as the segment of interest is a stable segment. When an instruction to start execution of the time phase detection function is provided in a freeze state, an end-diastole and an end-systole may be detected in the current segment of interest regardless of whether or not the current segment of interest is a stable segment. In any case, so long as the time phase detection function is in the ON state, after a change of segment of interest, a stable segment is selected as the segment of interest after change. A freeze state is a state in which transmission and reception are stopped. In a freeze state, a stored tomographic image is displayed, and a stored electrocardiac waveform is also displayed.

In an embodiment, when a going-back manipulation is performed, the selection control unit selects, as the segment of interest after change, a stable segment located before and closest to the segment of interest. On the other hand, when a going-forward manipulation is performed, the selection control unit selects, as the segment of interest after change, a stable segment located after and closest to the segment of interest. For example, when changes of segment of interest are repeated, jumps to new segments of interest are sequentially made. Every time the segment of interest is changed, detection of an end-diastole and an end-systole is performed again.

According to the above configuration, since there is no need for the user to designate or select stable segments, the user’s workload can be reduced. Further, according to the above configuration, it is possible to avoid performing cardiac function measurement based on an end-diastole and an end-systole detected in an inappropriate segment.

In an embodiment, during a real-time operation and in a freeze state, when an electrocardiac waveform is displayed, individual stable segments are displayed in an identifiable manner. For example, the individual stable segments are displayed in a specific color. On the electrocardiac waveform, in addition to the end-diastole marker and the end-systole marker, a time-phase-of-interest marker indicating a time phase of interest is displayed. An ultrasound image corresponding to the time phase of interest is displayed. During a real-time operation, ultrasound images are displayed in real time while ultrasound transmission and reception are carried out.

In an embodiment, the selection control unit has a function of identifying a best stable segment from among the stable segments, and selecting the best stable segment as an initial segment of interest or a segment of interest after change. According to this function, cardiac function measurement can be performed based on highly-reliable end-diastole and end-systole. The best stable segment is a segment which is judged as being most stable among the stable segments. For example, evaluation values may be calculated regarding the respective stable segments, and the stable segment corresponding to the best evaluation value may be determined as the best stable segment.

In an embodiment, the selection control unit has a function of identifying, from among the stable segments, a closest stable segment which is located closest to the current time phase of interest, and selecting the closest stable segment as an initial segment of interest or a segment of interest after change. According to this function, a segment that is located close to the time phase in which the user is interested and that is also appropriate in terms of cardiac function measurement can be used as the target of end-diastole and end-systole detection. In other words, it is possible to avoid a situation where there is selected a stable segment temporally located considerably away from the time phase in which the user is interested. Typically, the current time phase of interest is a time phase indicated by a time-phase-of-interest marker on a time axis. For example, at a point immediately after transition to a freeze state, the latest time phase is the current time phase of interest. When the user moves (i.e., shifts) the time-phase-of-interest marker, the time phase of interest is accordingly moved along the time axis.

In an embodiment, the determining unit determines stable segments during a real-time operation. The detection unit detects an end-diastole and an end-systole in a freeze state after the real-time operation. By continuously carrying out determination of stable segments during a real-time operation, stable segments can be recognized during the real-time operation, and the stable segments can be identified immediately after the real-time operation. Further, detection of an end-diastole and an end-systole can be promptly performed in a freeze state after the real-time operation.

In an embodiment, the stability criterion includes a primary determination criterion for determining a provisional stable segment based on durations of temporally consecutive segments, and a secondary determination criterion for determining that a duration of a provisional stable segment which has satisfied the primary determination criterion does not correspond to tachycardia or bradycardia. The determining unit includes a primary determining unit configured to apply the primary determination criterion to each segment, and a secondary determining unit configured to apply the secondary determination criterion to a provisional stable segment which has satisfied the primary determination criterion. The above-noted processor functions as the primary determining unit and the secondary determining unit.

According to the above configuration, a segment that is recognized as being stable but corresponds to tachycardia and a segment that is recognized as being stable but corresponds to bradycardia can be prevented from being used as a segment of interest. Other determination criteria such as a tertiary determination criterion may be added.

An electrocardiac waveform processing method according to an embodiment includes a defining step, a detecting step, a display processing step, a determining step, and a selection control step. In the defining step, a plurality of segments are defined, in units of heartbeat, in an electrocardiac waveform representing an electrocardiac signal obtained from an examinee. In the detecting step, based on the electrocardiac waveform, an end-diastole and an end-systole are detected in a segment of interest among the plurality of segments. In the display processing step, an end-diastole marker indicating the end-diastole in the segment of interest and an end-systole marker indicating the end-systole in the segment of interest are displayed in a case where the electrocardiac waveform is displayed together with an ultrasound image. In the determining step, from among the plurality of segments, each segment that satisfies a stability criterion is determined as a stable segment. In the selection control step, when a manipulation for changing the segment of interest is performed, selection of a segment of interest after change is controlled such that the segment of interest after change is selected from among the determined stable segments. When a manipulation for changing the segment of interest is performed, an end-diastole and an end-systole are detected in a specific stable segment, which is the segment of interest after change.

The above method is implemented as a hardware function or a software function. A program for executing the above method may be installed into an information processor via a network or a portable storage medium. The information processor includes an ultrasonic diagnosis apparatus. The information processor includes a non-transitory storage medium that stores the program.

### Details of Embodiments

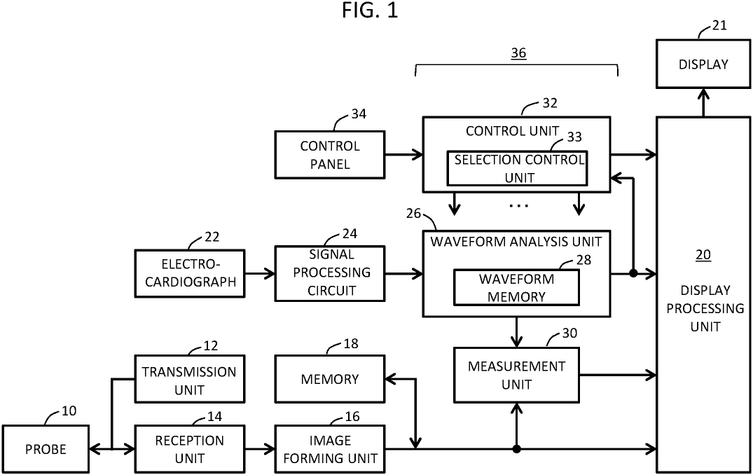

FIG. 1 shows an example configuration of an ultrasonic diagnosis apparatus according to an embodiment. This ultrasonic diagnosis apparatus is a medical apparatus to be provided in a medical institution such as a hospital and used for ultrasonic examination of examinees.

As shown in FIG. 1, the ultrasonic diagnosis apparatus comprises a probe 10. An ultrasound transmission/reception surface of the probe 10 is abutted against a surface of an examinee’s body. In that state, ultrasound waves are transmitted from the probe 10 to the interior of the living body, and reflected waves from the interior of the living body are received by the probe 10.

Specifically, the probe 10 has a transducer array composed of a plurality of transducers. An ultrasound beam is formed by the transducer array, and this ultrasound beam is electronically scanned. As electronic scan methods, an electronic linear scan method, an electronic sector scan method, and the like are known. As a result of the electronic scan of the ultrasound beam, a beam scan plane is formed. A two-dimensional transducer array may alternatively be provided in the probe 10, and a volume data may be obtained from the interior of the living body using the two-dimensional transducer array.

A transmission unit 12 is an electronic circuit that functions as a transmission beamformer, and a reception unit 14 is an electronic circuit that functions as a reception beamformer. At the time of transmission, a plurality of transmission signals are supplied in parallel from the transmission unit 12 to the transducer array, and a transmission beam is thereby formed. At the time of reception, a plurality of reception signals are output in parallel from the transducer array to the reception unit 14. In the reception unit 14, the reception signals are subjected to phase alignment and summing (i.e., delay and summing), and reception beam data are thereby generated.

A set of reception frame data is composed of a plurality of sets of reception beam data located serially along the electronic scan direction. Each set of reception beam data is composed of a plurality of sets of echo data located serially along the depth direction. Each set of reception frame data corresponds to a tomographic image. In a color flow mapping (CFM) mode, a set of reception frame data for formation of a tomographic image and a set of reception frame data for formation of a blood flow image are typically obtained alternately.

An image forming unit 16 serves to generate a display frame data array based on an input reception frame data array, and output the display frame data array. The display frame data array corresponds to tomographic images in the form of moving images. Each set of display frame data corresponds to a tomographic image in the form of a still image. The image forming unit 16 comprises a digital scan converter (DSC). The DSC is a kind of processor, and has functions such as a coordinate conversion function, an interpolation function, and a frame rate conversion function. The display frame data array is transmitted to a display processing unit 20.

A memory 18 is a so-called cine memory, and the display frame data array ranging over a certain period of time is stored into the memory 18. In a freeze state, the display frame data read out from the memory 18 are transmitted to the display processing unit 20 and a measurement unit 30. The memory 18 may be located upstream of the image forming unit 16.

The display processing unit 20 is composed of a processor. The display processing unit 20 has a graphic image generation function, an image compositing function, a color calculation function, and the like. In the display processing unit 20, images to be displayed on a display 21 are generated. In the present embodiment, the display processing unit 20 has a function of generating images of waveforms including an electrocardiac waveform (i.e., comprises a waveform image generator described later).

An electrocardiograph 22 comprises electrodes to be attached to the examinee. By means of the electrocardiograph 22, an electrocardiac signal (i.e., an ECG signal) is obtained from the examinee. The electrocardiac signal is transmitted to a waveform analysis unit 26 via a signal processing circuit 24.

The waveform analysis unit 26 is composed of a processor and a waveform memory 28. The waveform analysis unit 26 serves to analyze the waveform of the electrocardiac signal (i.e., the electrocardiac waveform). In the present embodiment, the waveform analysis unit 26 has a function of defining a plurality of heartbeat segments in the electrocardiac waveform, a function of determining, from among the plurality of heartbeat segments, each heartbeat segment that satisfies a stability criterion as a stable segment, a function of detecting an end-diastole and an end-systole in a selected heartbeat segment (i.e., a segment of interest), and the like.

The electrocardiac waveform ranging over a certain period of time is stored into the above-noted waveform memory 28. Time phases of waveform elements in the electrocardiac waveform stored into the waveform memory 28 are correlated to time phases of the sets of display frame data stored the memory 18.

The measurement unit 30 is composed of a processor. The measurement unit 30 serves to measure and calculate an evaluation value for evaluating the cardiac function. The evaluation value includes, for example, the ejection fraction (EF). The ejection fraction is calculated from the left ventricular volume at an end-diastole and the left ventricular volume at an end-systole. In order to calculate a reliable ejection fraction, it is necessary to increase the reliability of detected end-diastole and end-systole. In other words, it is necessary to detect an end-diastole and an end-systole in a stable heartbeat segment and not in an inappropriate heartbeat segment.

A control unit 32 serves to control operation of the elements shown in FIG. 1. The control unit 32 functions as a selection control unit 33. The selection control unit 33 controls selection of a segment of interest such that a stable segment is selected as the segment of interest. The segment of interest is a heartbeat segment that contains the time phase of interest. The time phase of interest is a time phase indicated by a time-phase-of-interest marker displayed on a screen. In the segment of interest, an end-diastole and an end-systole are detected. In the present embodiment, as a result of a control performed by the selection control unit 33, a stable segment is selected as the segment of interest at the time of a change of segment of interest.

The control unit 32 is composed of, for example, a processor that executes programs, which may be a CPU. As shown by reference numeral 36, the entirety of the control unit 32, waveform analysis unit 26, and measurement unit 30 may be implemented by a single processor. Furthermore, this processor may be caused to function as the display processing unit 20.

A control panel 34 includes switches, a trackball, a keyboard, and the like. In the present embodiment, the control panel 34 includes a go-back button and a go-forward button which will be described later, and also includes a button for causing execution of the stable segment determination function, a button for causing execution of the time phase detection function, and the like. The display 21 is composed of an organic EL device, a liquid crystal display, or the like.

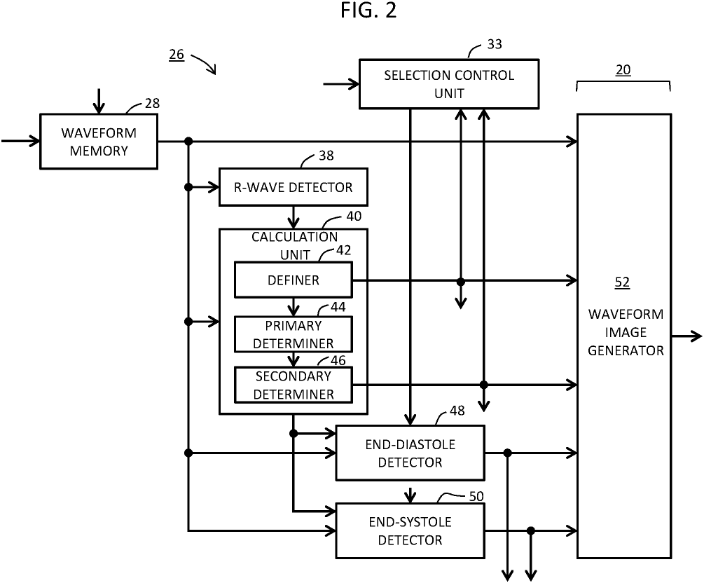

FIG. 2 shows an example configuration of the waveform analysis unit 26. FIG. 2 also shows the selection control unit 33 within the control unit, and a waveform image generator 52 within the display processing unit 20. An electrocardiac waveform is stored into the memory 28. At the time of a real-time operation, an input electrocardiac waveform is stored into the waveform memory 28, and is further supplied in parallel to an R-wave detector 38, a calculation unit 40, an end-diastole detector 48, and an end-systole detector 50.

The R-wave detector 38 serves to detect each R-wave (more specifically, the apex of each R-wave) contained in the electrocardiac waveform. The calculation unit 40 comprises a definer 42, a primary determiner 44, and a secondary determiner 46. The definer 42 functions as a defining unit or a defining means. The definer 42 divides or segments the electrocardiac waveform based on the detected R-waves, and thereby defines heartbeat segments in the electrocardiac waveform.

The primary determiner 44 and the secondary determiner 46, as a whole, function as a determining unit or a determining means. The primary determiner 44 serves to determine whether or not each heartbeat segment satisfies a primary determination criterion (i.e., a stability criterion). A heartbeat segment that satisfies the primary determination criterion is determined as a provisional stable segment. For example, in the primary determiner 44, durations of heartbeat segments that are consecutive on the time axis are compared with each other. The primary determination criterion will be described later in detail.

The secondary determiner 46 serves to determine whether or not a provisional stable segment satisfies a secondary determination criterion (i.e., a non-tachycardia criterion and a non-bradycardia criterion). A provisional stable segment that satisfies the secondary determination criterion is determined as a stable segment. The secondary determiner 46 functions as a tachycardia filter and a bradycardia filter. For example, in the secondary determiner 46, reference is made to the duration of the provisional stable segment. The secondary determination criterion will be described later in detail.

In the present embodiment, the calculation unit 40 has a function of determining, during a real-time operation, whether or not each heartbeat segment is a stable segment. When this function is executed, detection of an end-diastole and an end-systole can be performed promptly in a freeze state after the real-time operation. Determination of stable segments may also be carried out in a freeze state.

The end-diastole detector 48 and the end-systole detector 50, as a whole, function as a detection unit or a detecting means. Every time the segment of interest is changed, the end-diastole detector 48 and the end-systole detector 50 detect an end-diastole and an end-systole in the new segment of interest. The detection methods thereof will be described later. The selection control unit 33 controls the operations of the end-diastole detector 48 and the end-systole detector 50.

When a manipulation for changing the segment of interest is performed, the selection control unit 33 restricts selection of segment of interest such that a stable segment becomes the new segment of interest after change; i.e., such that a segment that is not a stable segment does not become the new segment of interest after change. Segment information, stable segment information, and the like are transmitted from the calculation unit 40 to the selection control unit 33.

The waveform image generator 52 serves to generate a waveform image in the form of a graphic image. The waveform image includes an electrocardiac waveform, a set of markers, and the like. When determination of stable segments is executed, the individual stable segments are identifiably displayed in the electrocardiac waveform. For example, when the electrocardiac waveform is displayed in a first color, each stable segment is displayed in a second color different from the first color. The set of markers, which is displayed in a freeze state, includes a time-phase-of-interest marker, an end-diastole marker, an end-systole marker, and the like. In a freeze state, a tomographic image (i.e., a still image) corresponding to the time phase of interest is displayed. Stored moving images may also be reproduced in a freeze state.

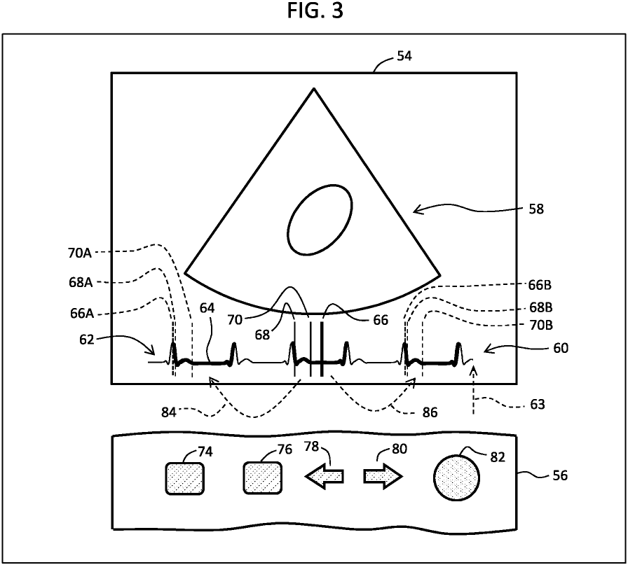

FIG. 3 shows a freeze state. On a display screen 54, a tomographic image 58 in the form of a still image is displayed, and a waveform image 60 including an electrocardiac waveform 62 is displayed. A control panel 56 includes a button 74 for instructing start of execution of the function to determine stable segments, a button 76 for instructing start of execution of the function to detect an end-diastole and an end-systole, a go-back button 78, a go-forward button 80, a freeze button 82, and the like. These buttons may be configured as virtual buttons displayed on a touchscreen panel.

When the button 74 is manipulated during a real-time operation, stable segments are sequentially determined while performing the real-time operation. In the present embodiment, pressing of the button 76 is not allowed during real-time operation. At the point when the button 82 is manipulated, a transition is made from the real-time operation state to a freeze state. In the freeze state, the button 74 and the button 76 can be manipulated. In a case where the button 74 has already been manipulated during the real-time operation and in a case where the button 74 is manipulated in the freeze state, the button 76 may be automatically turned on. It may be configured such that manipulation of the button 76; that is, instruction of execution of the time phase detection function, is allowed during the real-time operation.

In the electrocardiac waveform 62, the heartbeat segments depicted in bold lines 64 are stable segments. Reference numeral 63 denotes the first time phase of interest (i.e., the initial time phase of interest) at the point of transition to the freeze state. In the example shown in drawing, a time-phase-of-interest marker 66, which corresponds to a cursor, indicates the time phase of interest, which is currently of interest. In FIG. 3, the time-phase-of-interest marker 66 is located in the heartbeat segment (i.e., the stable segment) located at the center on the time axis (i.e., the horizontal axis). In this heartbeat segment, the end-diastole and the end-systole have already been detected, and an end-diastole marker 68 an end-systole marker 70 are displayed in this heartbeat segment. When the time-phase-of-interest marker 66 is shifted along the time axis using a trackball or the like, the tomographic image content is changed. That is, the tomographic image content is changed according to the changed time phase of interest. When the time-phase-of-interest marker 66 is aligned with the end-diastole marker 68, a tomographic image corresponding to the end-diastole is displayed. When the time-phase-of-interest marker 66 is aligned with the end-systole marker 70, a tomographic image corresponding to the end-systole is displayed. Movement of the time-phase-of-interest marker 66 may be restricted such that the movement can only be made to within stable segments, or the time-phase-of-interest marker 66 may be allowed to be moved freely.

When the go-back button 78 is manipulated in the state shown in FIG. 3, selection of segment of interest jumps to a stable segment located before and closest to the current segment of interest on the time axis (see reference numeral 84). In the example shown in FIG. 3, the stable segment located on the left becomes the new segment of interest. In this case, a time-phase-of-interest marker 66A jumps to the leading edge of the new segment of interest. A tomographic image corresponding to the time phase of this leading edge is displayed. In the new segment of interest, the end-diastole and the end-systole are automatically detected, and an end-diastole marker 68A an end-systole marker 70A indicating those detected end-diastole and end-systole are immediately displayed.

When the go-forward button 80 is manipulated in the state shown in FIG. 3, selection of segment of interest jumps to a stable segment located after and closest to the current segment of interest on the time axis (see reference numeral 86). In the example shown in FIG. 3, the stable segment located on the right becomes the new segment of interest. In this case, a time-phase-of-interest marker 66B jumps to the leading edge of the new segment of interest. A tomographic image corresponding to the time phase of this leading edge is displayed. In the new segment of interest, the end-diastole and the end-systole are automatically detected, and an end-diastole marker 68B and an end-systole marker 70B indicating those detected end-diastole and end-systole are immediately displayed. Every time the go-back button 78 or the go-forward button 80 is manipulated, the above-described operation is automatically executed.

According to the present embodiment, at the time of a change of segment of interest, a stable segment becomes the segment of interest after change; i.e., selection of the segment of interest is limited to stable segments. Accordingly, detection of an end-diastole and an end-systole in a stable segment is ensured without having the user judge whether or not each heartbeat segment is a stable segment. In other words, appropriate preparation or precondition for cardiac function measurement can be achieved.

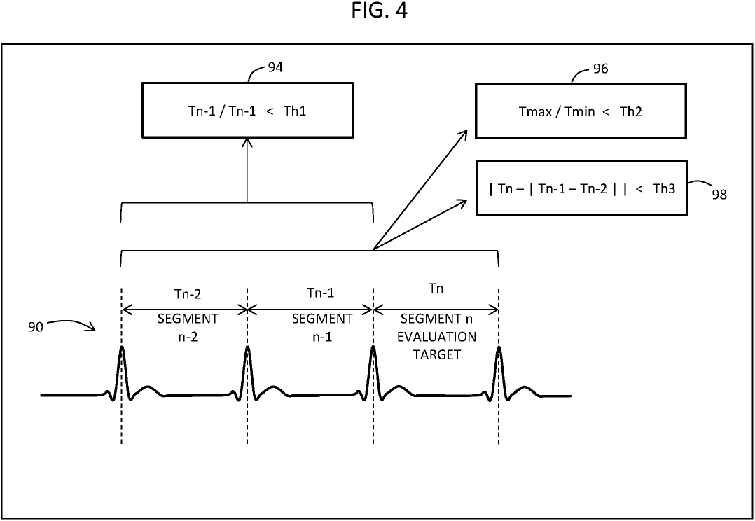

FIG. 4 shows a small number of primary determination criteria. The electrocardiac waveform 90 contains three heartbeat segments n, n-1, and n-2, which are consecutive on the time axis. Among these, the heartbeat segment n is the evaluation target segment.

A first primary determination criterion 94 is a criterion for determining whether or not the evaluation target segment n is a stable segment (i.e., a provisional stable segment) based on the duration Tn-1 of the heartbeat segment n-1 located one before the evaluation target segment n and the duration Tn-2 of the heartbeat segment n-2 located two before the evaluation target segment n. Specifically, the evaluation target segment n is determined as a stable segment when (Tn-1)/(Tn-2) is less than a threshold value Th1. This determination is based on the presumption that when there is not much difference between the durations of two temporally consecutive heartbeat segments, the heartbeat segment that occurs immediately after those two heartbeat segments is a stable segment. According to the first primary determination criterion, it is possible to evaluate the evaluation target segment n even at a point before the evaluation target segment n has reached its end.

A second primary determination criterion 96 and a third primary determination criterion 98 are criteria for determining whether or not the evaluation target segment n is a stable segment (i.e., a provisional stable segment) based on the durations of the three heartbeat segments including the evaluation target segment n. In the case of applying the second primary determination criterion 96, the maximum value Tmax and the minimum value Tmin are identified from among the durations of the three heartbeat segments. When the ratio Tmax/Tmin is less than a threshold value Th2, the evaluation target segment n is determined as a stable segment. In the case of applying the third primary determination criterion 98, the evaluation target segment n is determined as a stable segment when |Tn-|(Tn-1)-(Tn-2)|| is less than a threshold value Th3.

When employing the second primary determination criterion 96 and the third primary determination criterion 98, evaluation of the evaluation target segment n can be performed only after the evaluation target segment n has reached its end. Meanwhile, since the duration of the evaluation target segment n can be taken into consideration, it is possible to more accurately determine whether or not the evaluation target segment n is a stable segment.

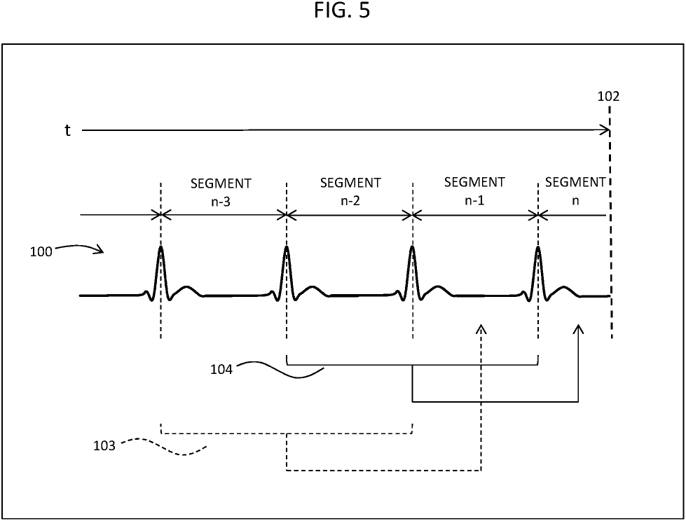

In a case where the first primary determination criterion is employed, the determination is performed during a real-time operation as shown in FIG. 5. Specifically, in an electrocardiac waveform 100, regarding every set of three temporally consecutive heartbeat segments including the latest heartbeat segment, whether or not the most recent heartbeat segment is a stable segment (i.e., a provisional stable segment) is determined based on the durations of the two heartbeat segments other than the most recent heartbeat segment (see reference numerals 103 and 104). Here, reference numeral 102 denotes the current time phase.

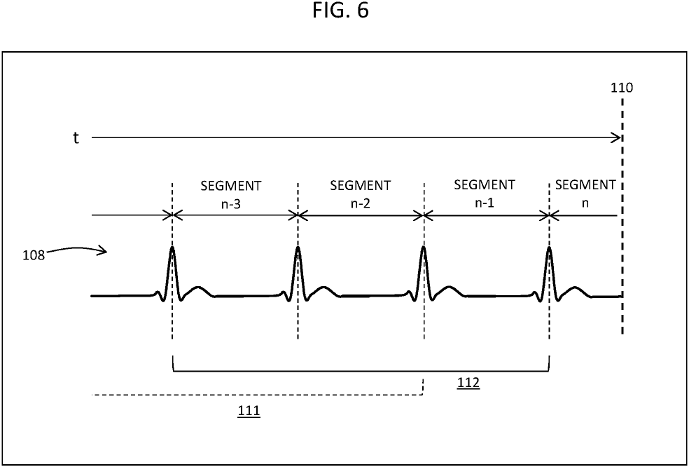

In a case where the second or the third primary determination criterion is employed, the determination is performed during a real-time operation as shown in FIG. 6. Specifically, in an electrocardiac waveform 108, regarding every set of three temporally consecutive heartbeat segments excluding the latest heartbeat segment, whether or not the most recent heartbeat segment is a stable segment is determined based on the durations of the three heartbeat segments (see reference numerals 111 and 112). Here, reference numeral 110 denotes the current time phase.

The secondary determination criterion applied in the above-noted secondary determination is a criterion for eliminating heartbeat segments that correspond to tachycardia and heartbeat segments that correspond to bradycardia. In the present embodiment, the secondary determination criterion is satisfied when the duration of a provisional stable segment is greater than a threshold value Th4 and less than a threshold value Th5. In other words, in that case, the provisional stable segment that has satisfied the primary determination criterion is determined as a stable segment. Instead of the duration of the provisional stable segment, other secondary determination criteria equivalent thereto for evaluating heartbeats may be used. In any case, by evaluating the size of the provisional stable segment, it is possible to avoid incorrectly determining an inappropriate heartbeat segment as a stable segment.

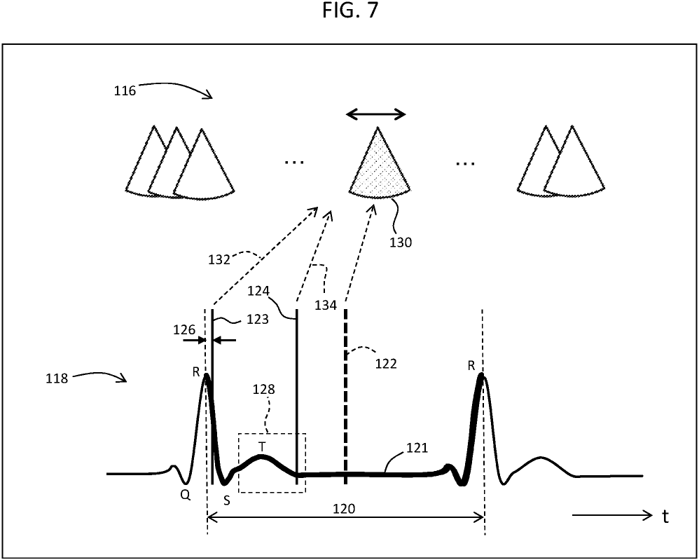

FIG. 7 illustrates a method of detecting an end-diastole and an end-systole. FIG. 7 shows a freeze state. In FIG. 7, an electrocardiac waveform 118 is shown in the lower part, and an array of tomographic images 116 is shown in the upper part.

A segment of interest 120 containing a time-phase-of-interest marker 122 is a stable segment. The segment of interest 120 is identifiably displayed (see reference numeral 121). A tomographic image 130 corresponding to the time phase of interest is displayed. The segment of interest 120 is a segment between two temporally adjacent R-waves. The end-diastole is estimated to be at a point after a certain period of time 126 from the R-wave at the leading edge of the segment of interest 120, and an end-diastole marker 123 is displayed at a position corresponding to that time phase. Further, the end-systole is estimated based on an analysis of the T-wave in the segment of interest 120 (see reference numeral 128), and an end-systole marker 124 is displayed at a position corresponding to that time phase. For example, the time phase at which the slope at the end of the T-wave becomes zero is determined as the end-systole. The end-diastole and the end-systole may be detected by methods other than those described above.

When the time-phase-of-interest marker 122 is aligned with the end-diastole marker 123, a tomographic image corresponding to the end-diastole is selected from among the array of tomographic images 116, and the selected tomographic image is displayed (see reference numeral 132). When the time-phase-of-interest marker 122 is aligned with the end-systole marker 124, a tomographic image corresponding to the end-systole is selected from among the array of tomographic images 116, and the selected tomographic image is displayed (see reference numeral 134).

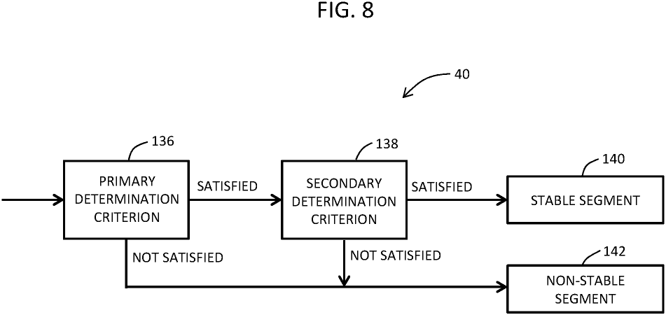

FIG. 8 shows the operation for determining a stable segment; i.e., the operation of the primary determiner and the second determiner. A heartbeat segment that satisfies both of a primary determination criterion 136 and a second determination criterion 138 is determined as a stable segment 140. A heartbeat segment that does not satisfy both of the primary determination criterion 136 and the second determination criterion 138 is determined as a non-stable segment.

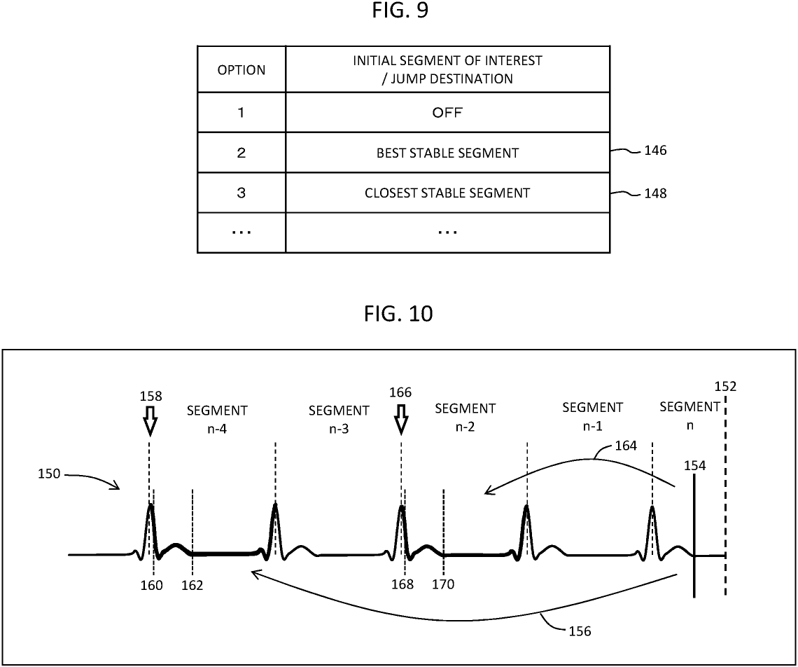

FIG. 9 shows a small number of options concerning the function of detecting end-diastole and end-systole (i.e., the time phase detection function). At a point when the time phase detection function is turned on, an already-selected option is executed. For example, in a case where the time phase detection function is automatically turned on at a point of transition to a freeze state, the initial segment of interest is selected according to the already-selected option. Further, in a case where the time phase detection function is manually turned on in a freeze state and a manipulation for changing the segment of interest is performed, the jump destination to which the time-phase-of-interest marker jumps; i.e., the segment of interest after change, is selected according to the already-selected option.

When option 2 denoted by reference numeral 146 is selected, the best stable segment is used as the initial segment of interest or the jump destination. An evaluation value indicating a stability level is calculated for every stable segment, and the stable segment corresponding to the best evaluation value is determined as the best stable segment. As the evaluation value, the values shown in FIG. 4 (on the left side of the respective mathematical formulas) may be employed. According to option 2, the most reliable stable segment can be used as the target of measurement.

When option 3 denoted by reference numeral 148 is selected, the closest stable segment is used as the initial segment of interest or the jump destination. The closest stable segment is the stable segment that is located before or after the current time phase of interest and closest to the time phase of interest or the segment of interest. At a point of transition to a freeze state, since the latest time phase is used as the current time phase of interest, the most recent stable segment as determined on the basis of that time phase is identified as the closest stable segment. In a freeze state, when the time-phase-of-interest marker is located at an arbitrary position, the time phase of interest or the segment of interest is used as the basis for identifying the closest stable segment. According to option 3, the stable segment that is located closest to the point of transition to the freeze state can be used as the target of measurement, or the stable segment that is located closest to the current time phase of interest can be used as the target of measurement.

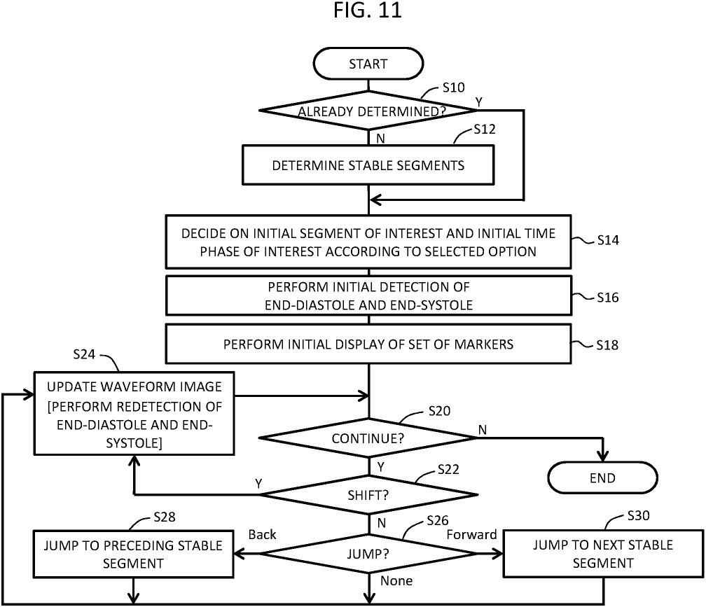

FIG. 10 shows an example operation according to option 2 and an example operation according to option 3. In an electrocardiac waveform 150, the latest time phase 152 is the time phase at the point of transition to a freeze state. In FIG. 10, the current time phase of interest is the time phase identified by a time-phase-of-interest marker 154. Assuming that option 2 is currently selected, when the time phase detection function is turned on, the best stable segment n-4 becomes the new segment of interest as denoted by reference numeral 156, and the leading edge thereof 158 becomes the new time phase of interest. The time-phase-of-interest marker is displayed at that leading edge.

On the other hand, assuming that option 3 is currently selected, when the time phase detection function is turned on, the closest stable segment n-2 becomes the new segment of interest as denoted by reference numeral 164, and the leading edge 166 thereof becomes the new time phase of interest. The time-phase-of-interest marker is displayed at the leading edge 166.

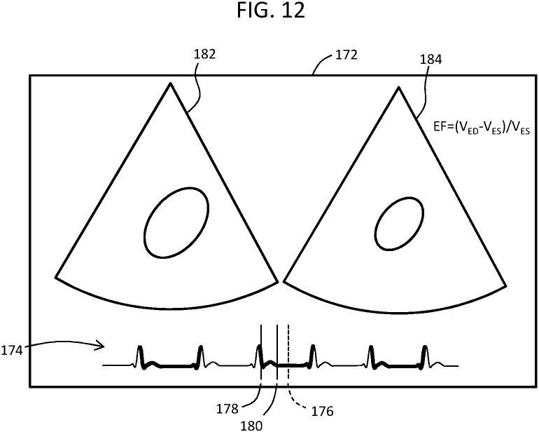

FIG. 11 shows an example operation, or more particularly, an example operation after a transition to a freeze state, of the ultrasonic diagnosis apparatus according to the present embodiment. In S10, it is judged whether or not stable segment determination has been executed during real-time operation. When not executed, in S12, it is determined whether or not each heartbeat segment constituting the electrocardiac waveform is a stable segment. Prior to that, as necessary, a button for causing execution of the stable segment determination function is manipulated, and a button for causing execution of the function of automatically detecting end-diastole and end-systole is manipulated.

In S14, the initial segment of interest and the initial time phase of interest are identified according to the selected option. When option 0 is selected, the initial time phase of interest is the latest time phase at the point of the transition to the freeze state, and the initial segment of interest is the heartbeat segment containing the latest time phase. When option 1 is selected, the best stable segment becomes the segment of interest, and the leading edge thereof becomes the time phase of interest. When option 2 is selected, the closest stable segment as determined with reference to the latest time phase becomes the segment of interest, and the leading edge thereof becomes the time phase of interest.

In S18, an end-diastole and an end-systole are detected in the segment of interest. Subsequently, the set of markers; namely, the time-phase-of-interest marker, the end-diastole marker, and the end-systole marker, are displayed within the segment of interest. In S20, whether or not to continue the present processing is judged.

In S22, it is determined whether or not there has been a manipulation for shifting the time-phase-of-interest marker. When the manipulation has been performed, in S24, the waveform image is updated, and at the same time, the tomographic image is updated. When the time-phase-of-interest marker has entered an adjacent heartbeat segment, an end-diastole and an end-systole are detected in that new segment of interest.

In S26, it is determined whether or not there has been a manipulation of the go-back button or the go-forward button. When a manipulation of the go-back button is determined in S26, in S28, the stable segment located before and closest to the current segment of interest becomes the new segment of interest after change. In other words, selection of segment of interest jumps in the past direction. After that, in S24, the waveform image and the tomographic image are updated. On the other hand, when a manipulation of the go-forward button is determined in S26, in S30, the stable segment located after and closest to the current segment of interest becomes the new segment of interest after change. In other words, selection of segment of interest jumps in the future direction. After that, in S24, the waveform image and the tomographic image are updated.

As such, according to the operation according to the present embodiment, since a stable segment becomes the new segment of interest when there has been a going-back manipulation or a going-forward manipulation, detection of end-diastole and end-systole in a stable segment is ensured. Accordingly, it is possible to obtain the advantage that the user is not required to judge whether or not each heartbeat segment is a stable segment. Further, it is possible to prevent use, for performing cardiac measurement, of an end-diastole and an end-systole detected in an inappropriate heartbeat segment.

In FIG. 12, on a display screen 172, two tomographic images 182, 184 are displayed, and a waveform image 174 is also displayed. In the electrocardiac waveform included in the waveform image 174, a specific stable segment is the segment of interest. Within this segment of interest, a time-phase-of-interest marker 176, an end-diastole marker 178, and an end-systole marker 180 are displayed. The tomographic image 182 is a tomographic image corresponding to the end-diastole indicated by the end-diastole marker 178, and the tomographic image 184 is a tomographic image corresponding to the end-systole indicated by the end-systole marker 180. An evaluation value is calculated by performing measurement based on the tomographic images 182, 184. Since the end-diastole and the end-systole are detected in a stable segment, the evaluation value can be calculated based on two appropriate tomographic images. Waveform images may be displayed separately for the respective tomographic images 182, 184.

1. An ultrasonic diagnosis apparatus comprising a processor, wherein
the processor is configured to:
form an ultrasound image based on reception data obtained from an examinee;
define, in units of heartbeat, a plurality of segments in an electrocardiac waveform representing an electrocardiac signal obtained from the examinee;
detect, based on the electrocardiac waveform, an end-diastole and an end-systole in a segment of interest among the plurality of segments; and
in a case where the electrocardiac waveform is displayed together with the ultrasound image, display an end-diastole marker indicating the end-diastole in the segment of interest and an end-systole marker indicating the end-systole in the segment of interest, and
the processor is further configured to:
determine, from among the plurality of segments, each segment that satisfies a stability criterion as a stable segment;
when a manipulation for changing the segment of interest is performed, control selection of a segment of interest after change such that the segment of interest after change is selected from among stable segments determined; and
when a manipulation for changing the segment of interest is performed, detect an end-diastole and an end-systole in a specific stable segment, which is the segment of interest after change.

2. The ultrasonic diagnosis apparatus according to claim 1, wherein
the processor is configured to:
when a going-back manipulation is performed, select, as the segment of interest after change, a stable segment located before and closest to the segment of interest; and
when a going-forward manipulation is performed, select, as the segment of interest after change, a stable segment located after and closest to the segment of interest.

3. The ultrasonic diagnosis apparatus according to claim 1, wherein
the processor has a function of identifying a best stable segment from among the stable segments, and selecting the best stable segment as an initial segment of interest or the segment of interest after change.

4. The ultrasonic diagnosis apparatus according to claim 1, wherein
the processor has a function of identifying, from among the stable segments, a closest stable segment which is located closest to the current time phase of interest, and selecting the closest stable segment as an initial segment of interest or the segment of interest after change.

5. The ultrasonic diagnosis apparatus according to claim 1, wherein
the processor is configured to:
determine the stable segments during a real-time operation; and
in a freeze state after the real-time operation, detect the end-diastole and the end-systole.

6. The ultrasonic diagnosis apparatus according to claim 1, wherein
the stability criterion includes:
a primary determination criterion for determining a provisional stable segment based on durations of temporally consecutive segments; and
a secondary determination criterion for determining that a duration of a provisional stable segment which has satisfied the primary determination criterion does not correspond to tachycardia or bradycardia, and
the processor is configured to:
apply the primary determination criterion to each of the segments, and
apply the secondary determination criterion to a provisional stable segment which has satisfied the primary determination criterion.

7. An electrocardiac waveform processing method, including:
a step of defining, in units of heartbeat, a plurality of segments in an electrocardiac waveform representing an electrocardiac signal obtained from an examinee;
a step of detecting, based on the electrocardiac waveform, an end-diastole and an end-systole in a segment of interest among the plurality of segments; and
a step of displaying an end-diastole marker indicating the end-diastole and an end-systole marker indicating the end-systole in a case where the electrocardiac waveform is displayed together with an ultrasound image, wherein
the method further includes:
a step of determining, from among the plurality of segments, each segment that satisfies a stability criterion as a stable segment; and
a step of, when a manipulation for changing the segment of interest is performed, controlling selection of a segment of interest after change such that the segment of interest after change is selected from among the determined stable segments, and
when a manipulation for changing the segment of interest is performed, an end-diastole and an end-systole are detected in a specific stable segment, which is the segment of interest after change.
